(19) United States
(12) Patent Application Publication
(10) Pub. No.: US 2005/0214219 A1
(43) Pub. Date: Sep. 29, 2005

(54) 24p3 receptors and uses thereof

(76) Inventors: Michael Green, Boylston, MA (US); Laxminarayana Devireddy, Worcester, MA (US); Claude Gazin, Shrewsbury, MA (US)

Correspondence Address: FISH & RICHARDSON PC, P.O. BOX 1022, MINNEAPOLIS, MN, 55440-1022, US

(21) Appl. No.: 10/995,796

(22) Filed: Nov. 22, 2004

Related U.S. Application Data
(60) Provisional application No. 60/523,948, filed on Nov. 21, 2003.

(51) Int. Cl. A61K 49/00; G01N 33/53; G01N 33/567

(52) U.S. Cl. 424/9.2; 435/7.2

(57) ABSTRACT

A receptor for 24p3 has been identified. The receptor has been found to play a role in both iron transport and apoptosis. Accordingly, compounds that modulate the expression or activity of a 24p3R are useful for modulating iron transport and apoptosis.

### CROSS-REFERENCE TO RELATED APPLICATIONS

This application claims the benefit of the filing date of U.S. Ser. No. 60/523,948, filed Nov. 21, 2003. The contents of this prior application are hereby incorporated by reference in their entirety.

### TECHNICAL FIELD

The invention relates to apoptosis, inflammation, and iron transport.

### BACKGROUND

Lipocalins are a family of secreted proteins that are present in organisms from bacteria to humans and can bind small molecular weight ligands. The lipocalin mouse 24p3 has been implicated in diverse physiological responses including apoptosis, iron uptake, and differentiation. 24p3 homologs have been identified in various species including mice and rats, and are also referred to as neutrophil gelatinase associated lipocalin (NGAL). 24p3 in mice is known by various designations, including NGAL, 24p3 protein, SIP24, P25, lipocalin 2, and uterocalin. NGAL in rats is known as NGAL or alpha 2-microglobulin. NGAL/24p3 increases 7 to 10 fold in cultured mouse kidney cells in response to viral infection (Hraba-Renevey et al., 1989, Oncogene, 4:601-608) and is a major secretory product of lipopolysaccharide-stimulated, cultured mouse macrophages (Meheus et al., 1993, J. Immunol., 151:1535-1547).

NGAL/24p3 is a positive acute phase protein. It has been suggested that this protein is a scavenger of bacterial products at sites of inflammation (Nielsen et al., 1996, Gut, 38:414-420). It has also been suggested that NGAL/24p3 has an immunomodulatory function involving the binding of lipophilic inflammatory mediators (Bundgaard et al., 1994, Biochem. Biophys. Res. Comm., 202:1468-1475). NGAL/24p3 is synthesized constitutively at a particular developmental point during the maturation of granulocyte precursors in the bone marrow (Borregaard et al., 1995, Blood, 85:812-817). In addition, NGAL/24p3 synthesis can be induced in epithelial cells under certain conditions such as inflammation and malignancy (Neilsen et al., supra; Bartsch et al., 1995, FEBS Lett., 357:255-259; Bundgaard et al., supra). NGAL/24p3 is also involved in a two-stage, transcriptionally regulated apoptotic pathway. In the first stage, IL-3 withdrawal results in transcriptional activation of an NGAL/24p3 gene followed by synthesis and secretion of the protein. In the second stage, secreted NGAL/24p3 induces apoptosis in lymphoid cells by an autocrine mechanism (Devireddy et al., 2001, Science, 293:829-834).

The cell surface receptor for 24p3 has not been identified, and it is not known whether the distinct responses elicited by 24p3 result from a single or multiple receptors.

### SUMMARY

A complementary DNA (cDNA) encoding a murine 24p3 cell surface receptor (24p3R) has been isolated. Two forms of the murine receptor have been identified; a long form (24p3R-L) and a short form (24p3R-S). Rat and human homologs of the murine 24p3R have also been identified. The receptor has been found to mediate 24p3 activity in both apoptosis and iron transport. It has also been found that ectopic expression of murine 24p3R confers on cells the ability to undergo 24p3-dependent iron uptake and apoptosis, and that the cellular response depends on the iron content of the ligand.

In one aspect, the invention relates to methods of identifying a candidate compound that modulates an interaction between a 24p3 polypeptide and a 24p3 receptor (24p3R) polypeptide, by providing a sample including a 24p3R polypeptide; contacting the sample with a 24p3 polypeptide and a test compound, thereby providing a test sample; and determining whether binding between the 24p3 polypeptide and the 24p3R polypeptide is altered in the test sample compared to a control sample lacking the test compound. A difference in binding indicates that the test compound is a candidate compound that modulates an interaction between the 24p3 polypeptide and the 24p3R polypeptide.

In various embodiments, the test compound decreases or increases binding of 24p3 to the 24p3R, the test compound decreases or increases an inflammatory or apoptotic response, or the test compound decreases or increases iron transport.

In certain embodiments, the test sample is a cell. In alternative embodiments, the test sample is cell-free. In some embodiments, the test sample comprises a mammalian cell. In some embodiments, the test sample is derived from a mouse or a human. In some embodiments, the test compound is an antibody. In some embodiments, the test compound is a molecule that can bind iron. In some embodiments, the test compound is a 24p3 polypeptide.

In another aspect, the invention relates to methods of evaluating 24p3R expression in a cell. The method includes contacting the cell or a portion thereof with an anti-24p3R antibody, or the antigen-binding portion thereof, and detecting the antibody or antigen-binding portion. In some embodiments, the cell is a mammalian cell. In some embodiments, the cell is a mouse cell. In alternative embodiments, the cell is a human cell.

The invention also relates to a method of inducing apoptosis in a cell that expresses 24p3R. The method includes administering to the cell an amount of iron-free 24p3 sufficient to induce apoptosis. In certain embodiments, the cell is a mammalian cell. In some embodiments, the cell is a mouse cell. In alternative embodiments, the cell is a human cell. In some embodiments, the cell is in vivo.

In another aspect, the invention relates to increasing levels of a 24p3R polypeptide in a cell. The method includes administering to the cell the 24p3R expression vector described above. In certain embodiments, the cell is a mammalian cell. In some embodiments, the cell is a mouse cell. In alternative embodiments, the cell is a human cell. In various embodiments, the cell is in vitro or in vivo.

In a further aspect, the invention relates to isolated nucleic acid molecules that consist of a nucleotide sequence that encodes a long form of a murine 24p3 receptor (24p3R-L) polypeptide having an amino acid sequence of SEQ ID NO:2, or the amino acid sequence of 24p3R-L with conservative amino acid substitutions. The invention also includes nucleic acids having the nucleotide sequence of 24p3R-L (SEQ ID NO:1). The invention also includes purified or isolated polypeptides having the amino acid sequence of SEQ ID NO:2 with from 0 to 10 conservative amino acid substitutions.

In various embodiments, the invention also includes expression vectors containing any of the nucleic acids described herein or nucleic acids encoding a long form of a 24p3R operably linked to an expression control sequence, a cultured cell including the expression vector, and a cultured cell transfected with the vector, or a progeny of the cell, such that the cell expresses the polypeptide.

In another aspect, the invention relates to purified antibodies that specifically bind to a 24p3R or a portion thereof, e.g., the N terminus or the C terminus of a 24p3R. In certain embodiments, the antibody specifically binds to the sequence CDHVPLLATPNPAL (SEQ ID NO:3). In some embodiments, the antibody is a polyclonal antibody. In other embodiments, the antibody is a monoclonal antibody.

As used herein, “24p3R polypeptide” or “24p3R” means a receptor polypeptide, the amino acid sequence of which meets at least one of the following criteria:
(a) it contains an amino acid sequence that has at least 80% sequence identity with a full-length, known wild-type 24p3R polypeptide (e.g., human, mouse, or rat), e.g., the murine 24p3R amino acid sequence set forth in FIG. 2, or homologs from other species such as rat (e.g., Genbank Accession No. NP_{—}803156) and human (Genbank Accession No. NP_{—}065105), or a portion or fragment of the full-length wild-type polypeptide that measurably induces apoptosis in a lymphoid cell;
(b) it contains an amino acid sequence consisting of a consensus sequence constructed by alignment of the murine, rat, and human sequences (SEQ ID NO:2, Genbank Accession No. NP_{—}803156, and Genbank Accession No. NP_{—}065105, respectively), with up to 30 conservative amino acid substitutions; and up to 20 amino acid deletions or non-conservative amino acid substitutions (in any combination); or
(c) it binds to a 24p3 polypeptide with an affinity at least 40% (e.g., 40%, 60%, 80%, 90%, 95%, or 98%) of the affinity of the wild-type human 24p3R.

A 24p3R can be isolated from a natural source or it can be produced by recombinant DNA methods.

As used herein, “24p3,” “24p3/NGAL”, or “24p3-like polypeptide” means a glycosylated or nonglycosylated polypeptide, the amino acid sequence of which is a naturally occurring, mature 24p3 amino acid sequence from any mammal (e.g., human, mouse, or rat) that is a ligand of a 24p3R. A 24p3 polypeptide can be isolated from a natural source or it can be produced by recombinant DNA methods. Examples of 24p3 polypeptides include polypeptides consisting of the amino acid sequences of Genbank Accession no. BC033089.1 (human 24p3), NP_{—}032517 (murine lipocalin 2), P30152 (rat NGAL). In general, the 24p3 used in methods described herein is a mature protein that can bind to a cognate 24p3R and elicit a 24p3R-mediated response such as those associated with iron transport or apoptosis. Other appropriate sequences besides those described herein are known in the art and can be used in the methods disclosed herein.

As used herein, “conservative amino acid substitution” means a substitution of an amino acid in a polypeptide within an amino acid family. Families of amino acids are recognized in the art and are based on physical and chemical properties of the amino acid side chains. Families include the following: amino acids with basic side chains (e.g. lysine, arginine, and histidine); amino acids with acidic side chains (e.g., aspartic acid and glutamic acid); amino acids with uncharged polar side chains (e.g. glycine, asparagine, glutamine, serine, threonine, tyrosine, and cysteine); amino acids with nonpolar side chains (e.g. alanine, valine, leucine, isoleucine, proline, phenylalanine, methionine, and tryptophan); amino acids with branched side chains (e.g., threonine, valine, and isoleucine); and amino acids with aromatic side chains (e.g., tyrosine, phenylalanine, tryptophan, and histidine). An amino acid can belong to more than one family.

As used herein, “therapeutically effective” amount or dose refers to that amount of a compound sufficient to result in amelioration of at least one symptom of a disease or disorder, e.g., a leukemia, an autoimmune disorder, or hemochromatosis. Such symptoms are known in the art (for example, see Berkow et al., The Merck Manual, Merck Research Laboratories, N.J., 1992).

Unless otherwise defined, all technical and scientific terms used herein have the same meaning as commonly understood by one of ordinary skill in the art to which this invention belongs. In case of conflict, the present application, including definitions, will control. All publications, patents, and other references mentioned herein are incorporated by reference in their entirety.

Although methods and materials similar or equivalent to those described herein can be used in the practice or testing of the present invention, useful methods and materials are described below. The materials, methods and examples are illustrative only and not intended to be limiting. Other features and advantages of the invention will be apparent from the detailed description and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representation of a nucleic acid sequence encoding murine 24p3R-L (SEQ ID NO:1)

FIG. 2 is a representation of a predicted amino acid sequence encoding murine 24p3R-L (SEQ ID NO:2). Underlined sequences indicate predicted membrane spanning regions.

FIG. 3 is a set of micrographs of calcein-loaded HeLa cells. The first and third columns depict calcein fluorescence. The second and fourth columns depict control fluorescence due to DAPI staining.

FIG. 4 is a gel that depicts expression levels of 24p3, 24p3R, and controls as measured by RT-PCR and immunoblot analysis.

FIG. 5 is a gel that depicts expression levels of 24p3, 24p3R, and control GAPDH on addition of the BCR-ABL inhibitor STI.

### DETAILED DESCRIPTION

We describe herein a new murine 24p3 receptor (24p3R). This receptor has both a long and short form (24p3R-L and 24p3R-S, respectively). The term “24p3R” as used herein refers to either murine form as well as other homologous forms of the receptor described herein from other mammals (e.g., human, mouse, rat). We have discovered that the polypeptides we now refer to as 24p3R are cell surface receptors that are involved in both (i) a transferrin-independent iron uptake pathway that occurs in parenchymal tissues and (ii) apoptosis. The true nature of this class of polypeptides as cell surface receptors with these unique characteristics was not known prior to the present invention. We have also found that iron uptake and apoptosis are distinguishable responses dependent upon the iron content of the ligand. Iron-loaded 24p3 (referred to herein as holo-24p3) increases intracellular iron concentration and fails to induce apoptosis whereas iron-lacking 24p3 (referred to herein as apo-24p3) reduces intracellular iron levels and induces apoptosis.

On the basis of the results described herein, distinct activities elicited by 24p3 have been identified. Holo-24p3 binds to 24p3R, is internalized and releases its bound iron thereby increasing intracellular iron concentration. By contrast, apo-24p3 binds to 24p3R, is internalized and chelates iron thereby reducing intracellular iron concentration, resulting in apoptosis. The iron-load state of 24p3 (i.e., iron-loaded or iron-lacking) is a critical determinant of its activity.

24p3R binds 24p3 and is expressed early in murine development, when an iron transport pathway alternative to the transferrin pathway is thought to operate. Thus, 24p3R has expected properties of a cell surface receptor involved in the non-transferrin iron uptake pathway.

24p3 (NGAL) is induced in epithelial cells during inflammation. Compounds that bind to 24p3R and inhibit the response to 24p3 are thus useful for ameliorating undesirable inflammation. 24p3/NGAL levels are also elevated in certain diseases (e.g., atherosclerosis and respiratory distress syndrome). Compounds that modulate the activity of 24p3R, e.g., by binding to the receptor and preventing 24p3R-mediated signaling associated with NGAL binding, are useful for treating such disorders. Iron overload (hemochromatosis) is another class of disorder that can be ameliorated by compounds that inhibit the iron transport activity of a 24p3R. Compounds that bind to the receptor and interfere with its ability to mediate iron transport are useful for preventing undesirable accumulation of iron in a cell.

In addition, 24p3R maps to a chromosomal location in humans (14q11.2) that is believed to contain a tumor suppressor gene. The locus is mutated in several cancers (e.g., a nasopharyngeal carcinoma and a glioblastoma). Thus, compounds that modulate 24p3R can be useful for treating of such conditions. For example, if the receptor binds to its natural ligand in an abnormal manner, a compound can be sought that binds to the mutant form of the receptor and is able to restore at least part of the normal function. This restoration can be useful in treatment of a cancer if, for example, restoration of normal function restores growth-factor dependence on the cancer cell.

24p3R is useful in the treatment of various cancers. We show herein that the transport of apo-24p3 into the cell by 24p3R leads to a decrease in intracellular iron, which thus induces apoptosis. Thus, increasing 24p3R expression in cancer cells or delivering apo-24p3 to cancer cells that express 24p3R is an effective treatment for various cancers.

For example, 24p3 and 24p3R are implicated in cancers induced by the BCR-ABL fusion protein. The BCR-ABL fusion-protein is created through a translocation event that juxtaposes the c-abl tyrosine kinase gene on chromosome 9 with the breakpoint cluster region (bcr) on chromosome 22. Depending on the breakpoint site, this genetic structure can produce three different BCR-ABL fusion-proteins, p190 (190 kDa), p210 (210 kDa), or p230 (230 kDa), all of which contain the same portion of the c-Abl tyrosine kinase in the C-terminus but include different amounts of Bcr sequence at the N-terminus. These three forms of BCR-ABL are associated with distinct forms of leukemia. For example, the p210 form is responsible for the development of chronic myeloid leukemia (CML), whereas the p190 form leads to adult acute lymphoblastic leukemia (ALL).

The results described herein (Example 7), suggest that the downstream effectors of transformation by BCR-ABL include 24p3 and 24p3R. The p210 BCR-ABL fusion-protein is a constitutively active cytoplasmic kinase that is essential for hematopoietic cell transformation and is thought to exert its effect by interfering with cellular signal transduction pathways normally involved in the control of cell death and proliferation. The process of transformation by BCR-ABL is accompanied by growth factor independence, reduced susceptibility to apoptosis, and altered cellular motility. Interestingly, mice injected with BCR-ABL-expressing cells (i.e., BCR-ABL+cells) show atrophy of the bone marrow, characterized by a severe reduction in normal hematopoiesis and eventual death due to severe weight loss. Based on this finding, it has been proposed that BCR-ABL+cells secrete one or more factors that interfere with normal hematopoiesis and contribute to disease severity. As discussed in detail below, 24p3 expression is constitutively up-regulated in cells expressing BCR-ABL. As a result, conditioned medium from these cells induces apoptosis of normal hematopoietic cells. Imatinib mesylate (imatinib), a potent inhibitor of the BCR-ABL kinase, can suppress proliferation of BCR-ABL+cells in vitro and in vivo. Treatment of BCR-ABL expressing cells with imatinib leads to up-regulation of 24p3R and may in turn lead to increased apoptosis.

Based on the results described herein, the identification of a cell surface receptor for 24p3 provides a target for identifying compounds that modulate apoptosis and iron transport. Furthermore, increasing expression of 24p3R in cells of some cancers can lead to increased apoptosis of those cancer cells. For example, nucleic acids directing the expression of 24p3R can be introduced to cancer cells using gene therapy techniques (Chada et al., 2003, Curr. Opin. Mol. Ther., 5:463-74), including viral vectors (e.g., retroviral vectors or adenoviral vectors) and non-viral vectors (e.g., liposomes or molecular conjugates).

In another aspect, 24p3 is strongly overexpressed in osteoarthritic joints compared to joints of normal individuals (Zerega et al., 2000, Eur. J. Cell Biol., 79:165-172). Compounds that modulate the interaction between 24p3 and 24p3R are thus also useful in the treatment of osteoarthritis.

Screening Assays

In one aspect, the invention provides methods (also referred to herein as “screening assays”) for identifying modulators, i.e., candidate compounds or agents identified from one or more test compounds (e.g., proteins, peptides, peptidomimetics, peptoids, small inorganic molecules, small non-nucleic acid organic molecules, nucleic acids (e.g., anti-sense nucleic acids, siRNA, oligonucleotides, or synthetic oligonucleotides), or other drugs) that bind to a 24p3R, and have a stimulatory or inhibitory effect on, for example, activity. Compounds thus identified can be used to modulate the activity of a 24p3R in a therapeutic protocol, to elaborate the biological function of a 24p3/NGAL or 24p3R, or to identify compounds that disrupt normal 24p3/NGAL 24p3R interactions.

In one embodiment, assays are provided for screening test compound to identify those that can bind to a 24p3R or a portion thereof. In general, 24p3R polypeptides used in the assays described herein include at least the C terminal portion of a 24p3R or a biologically active portion thereof. Compounds that bind to a 24p3R can be tested for their ability to modulate an activity associated with 24p3R such as apoptosis, an inflammatory response, or iron transport.

The test compounds used in the methods described herein can be obtained using any of the numerous approaches in combinatorial library methods known in the art, including: biological libraries; peptoid libraries (libraries of molecules having the functionalities of peptides, but with a novel, non-peptide backbone, which are resistant to enzymatic degradation, but which, nevertheless, remain bioactive; see, e.g., Zuckermann et al., 1994, J. Med. Chem., 37:2678-2685); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer, or small molecule libraries of compounds (Lam, 1997, Anticancer Drug Des., 12:145).

Examples of methods for the synthesis of molecular libraries can be found in the art, for example in: DeWitt et al. (1993, Proc. Natl. Acad. Sci. USA, 90:6909; Erb et al., 1994, Proc. Natl. Acad. Sci. USA, 91:11422; Zuckermann et al., 1994, J. Med. Chem., 37:2678; Cho et al., 1993, Science, 261:1303; Carrell et al., 1994, Angew. Chem. Int. Ed. Engl., 33:2059; Carell et al., 1994, Angew. Chem. Int. Ed. Engl., 33:2061; and in Gallop et al., 1994, J. Med. Chem., 37:1233).

Libraries of compounds may be presented in solution (e.g., Houghten, 1992, Biotechniques, 13:412-421), or on beads (Lam, 1991, Nature, 354:82-84), chips (Fodor, 1993, Nature, 364:555-556), bacteria (Ladner, U.S. Pat. No. 5,223,409), spores (Ladner, U.S. Pat. No. 5,223,409), plasmids (Cull et al., 1992, Proc. Natl. Acad. Sci. USA, 89:1865-1869), or on phage (Scott and Smith, 1990, Science, 249:386-390; Devlin, 1990, Science, 249:404-406; Cwirla et al., 1990, Proc. Natl. Acad. Sci. USA, 87:6378-6382; Felici, 1991, J. Mol. Biol., 222:301-310; and Ladner supra).

In some embodiments, the assay is a cell-based assay in which a cell that expresses a 24p3R or biologically active portion thereof is contacted with a test compound, and the ability of the test compound to modulate 24p3R activity is determined, for example, by monitoring iron transport. The cell, for example, can be of mammalian origin, e.g., murine, rat, or human origin.

The ability of the test compound to modulate 24p3R binding to a ligand or substrate (e.g., a 24p3) can be evaluated, for example, by coupling the compound, e.g., the substrate, with a radioisotope or enzymatic label such that binding of the compound, e.g., the substrate, to 24p3R can be determined by detecting the labeled compound, e.g., substrate, in a complex. Alternatively, 24p3R can be coupled with a radioisotope or enzymatic label to monitor the ability of a test compound to modulate 24p3R binding to a 24p3 in a complex. For example, compounds (e.g., 24p3R substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 24p3R substrate) to interact with 24p3R with or without the labeling of any of the interactants can be evaluated. For example, a microphysiometer can be used to detect the interaction of a compound with 24p3R without labeling either the compound or the 24p3R (McConnell et al., 1992, Science 257:1906-1912). As used herein, a “microphysiometer” (e.g., Cytosensor®) is an analytical instrument that measures the rate at which a cell acidifies its environment using a light-addressable potentiometric sensor (LAPS). Changes in this acidification rate can be used as an indicator of the interaction between a compound and 24p3R.

In yet another embodiment, a cell-free assay is provided in which a 24p3R or biologically active portion thereof is contacted with a test compound and the ability of the test compound to bind to the 24p3R or biologically active portion thereof is evaluated. In general, biologically active portions of the 24p3R proteins to be used in the new assays include fragments that participate in interactions with 24p3 molecules, e.g., fragments that are in the extracellular domain of 24p3R.

Soluble and/or membrane-bound forms of isolated proteins (e.g., 24p3R or biologically active portions thereof) can be used in cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169 and Stavrianopoulos et al., U.S. Pat. No. 4,868,103). A fluorophore label on the first ‘donor’ molecule is selected such that its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 24p3R protein to bind to a target molecule (e.g., a 24p3) can be accomplished using real-time Biomolecular Interaction Analysis (BIA) (e.g., Sjolander et al., 1991, Anal. Chem., 63:2338-2345 and Szabo et al., 1995, Curr. Opin. Struct. Biol., 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. The target gene product can be anchored onto a solid surface, and the test compound, which is not anchored, can be labeled, either directly or indirectly, with detectable labels discussed herein.

It may be desirable to immobilize 24p3R, an anti-24p3R antibody, or its target molecule (e.g., a 24p3 polypeptide) to facilitate separation of complexed from uncomplexed forms of one or both of the proteins, as well as to accommodate automation of the assay. Binding of a test compound to a 24p3R protein, or interaction of a 24p3R protein with a target molecule in the presence or absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/24p3R fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein or 24p3R protein, and the mixture incubated under conditions conducive to complex formation (e.g., at physiological conditions for salt and pH). Following incubation, the beads or microtiter plate wells are washed to remove unbound components, the matrix immobilized in the case of beads, complex determined either directly or indirectly, for example, as described above. Alternatively, the complexes can be dissociated from the matrix, and the level of 24p3R binding or activity determined using standard techniques.

Other techniques for immobilizing either a 24p3R protein or a target molecule on matrices include using conjugation of biotin and streptavidin. Biotinylated 24p3R protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies that bind specifically to 24p3R polypeptides or target molecules, but do not interfere with binding of the 24p3R polypeptide to its target molecule (e.g., a 24p3 polypeptide). Such antibodies can be derivatized to the wells of the plate, and unbound target or 24p3R protein trapped in the wells by antibody conjugation. Methods for detecting such complexes, in addition to those described above for the GST-immobilized complexes, include immunodetection of complexes using antibodies reactive with the 24p3R protein or target molecule, as well as enzyme-linked assays which rely on detecting an enzymatic activity associated with the 24p3R protein or target molecule.

Alternatively, cell-free assays can be conducted in a liquid phase. In such an assay, the reaction products are separated from unreacted components, by any of a number of standard techniques, including but not limited to: differential centrifugation (for example, Rivas et al., 1993, Trends Biochem. Sci., 18:284-287); chromatography (gel filtration chromatography, ion-exchange chromatography); electrophoresis (e.g., Ausubel et al., eds., 1999, Current Protocols in Molecular Biology, J. Wiley: New York.); and immunoprecipitation (for example, Ausubel et al., eds., 1999, Current Protocols in Molecular Biology, J. Wiley: New York). Such resins and chromatographic techniques are known to those skilled in the art (e.g., Heegaard, 1998, J. Mol. Recognit., 11:141-148 and Hage et al., 1997, J. Chromatogr. B. Biomed. Sci. Appl., 699:499-525). Further, fluorescence energy transfer may also be conveniently utilized, as described herein, to detect binding without further purification of the complex from solution.

In one embodiment, the assay includes contacting the 24p3R protein or biologically active portion thereof with a known compound that binds to 24p3R to form an assay mixture, contacting the assay mixture with a test compound, and determining the ability of the test compound to interact with a 24p3R protein, wherein determining the ability of the test compound to interact with a 24p3R protein includes determining the ability of the test compound to preferentially bind to 24p3R or a biologically active portion thereof, or to modulate the activity of a target molecule, as compared to the known compound.

A 24p3R can, in vivo, interact with one or more cellular or extracellular macromolecules, such as proteins, e.g., a 24p3 or other proteins involved in 24p3R-mediated signaling in an apoptotic pathway or iron transport pathway. For the purposes of this discussion, such cellular and extracellular macromolecules are referred to herein as “binding partners.” Compounds that disrupt such interactions can be useful in regulating the activity of the 24p3R. Such compounds can include, but are not limited to, molecules such as antibodies, peptides, and small molecules as described herein. In general, target genes/products for use in this embodiment are the 24p3R genes/products identified herein. In an alternative embodiment, the invention provides methods for determining the ability of the test compound to modulate the activity of a 24p3R protein through modulation of the activity of a downstream effector of a 24p3R target molecule. In general, such assays are conducted after determining that a test compound can bind to a 24p3R and/or can interfere with the binding between a 24p3R and a 24p3. In such assays, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the 24p3R of interest and the binding partner (e.g., a 24p3 that can bind to the 24p3R) is prepared, under conditions and for a time sufficient, to allow the two products to form a complex. To test an inhibitory agent, the reaction mixture is provided in the presence and absence of the test compound. The test compound can be initially included in the reaction mixture, or can be added at a time subsequent to the addition of the target gene and its binding partner. Control reaction mixtures are incubated without the test compound or with a placebo. The formation of complexes between the target gene product and the binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant, but not normal, target gene products.

These assays can be conducted in a heterogeneous or homogeneous format. Heterogeneous assays involve anchoring either the 24p3R (or fragment thereof) or the binding partner (e.g., a 24p3 that can bind to the 24p3R) onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the 24p3R and the binding partner, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the 24p3R (or fragment thereof) or the interactive binding partner (e.g., a 24p3), is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex formation or that disrupt preformed complexes can be identified.

In some cases, a homogeneous assay can be used. For example, a preformed complex of a 24p3R (or fragment thereof) and the interactive binding partner (e.g., a 24p3) is prepared in that either the 24p3R or the binding partner is labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

24p3R Polypeptides

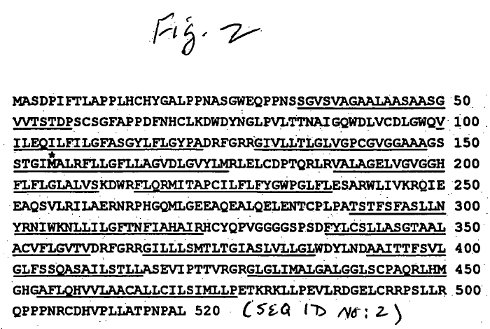

The 24p3R polypeptides used in the new methods can have, for example, the full-length murine 24p3R amino acid sequence shown in FIG. 2. The sequence can be that of the short or long form of the murine 24p3R, and can be the homologous short or long form of 24p3R polypeptides of non-murine species (e.g., rat such as Genbank accession no. NP_{—}803156 or human such as Genbank Accession No. NP_{—}065105). 24p3R polypeptides also include polypeptides that are variants or mutants of the murine, rat, or human short or long forms of 24p3R. For example, a suitable 24p3R polypeptide for binding to a 24p3 polypeptide can include at least one of the C-terminal portion of a 24p3R and the last two transmembrane regions of 24p3R (about amino acids 428-520 of murine 24p3R-L). Furthermore, those of skill in this field will recognize that within a species, natural amino acid polymorphisms may occur in the 24p3R amino acid sequences found in different individuals. Accordingly, the use of various naturally occurring forms of human, murine, or rat 24p3R polypeptides is within the scope of the invention.

The use of natural 24p3R polypeptides (or portions thereof) from various mammalian species to induce apoptosis in lymphoid cells is within the scope of the invention. The currently known natural 24p3R sequences, i.e., those from human, mouse, and rat, have highly conserved amino acid sequences (about 98% identity) and are expected to have activity in heterologous species. The invention therefore includes, for example, the use of a mouse or rat 24p3R polypeptide in a human patient, or the use of a human 24p3R polypeptide in a non-human animal undergoing veterinary treatment. The human 24p3R polypeptide, or a biologically active fragment thereof, can, of course, be used in human patients as well. In some embodiments, a chimeric 24p3R polypeptide is used, e.g., an artificial 24p3R polypeptide formed by replacing a portion of a human 24p3R amino acid sequence with a corresponding portion of an 24p3R amino acid sequence from another species.

A consensus sequence can be constructed using identified 24p3R amino acid sequences (e.g., SEQ ID NO:2, rat 24p3R (Genbank accession no. NP_{—}803156), and human 24p3R (Genbank Accession No. NP_{—}065105). The consensus sequence, wherein each amino acid position represents an amino acid or a gap from an alignment of identified 24p3R sequences, will induce apoptosis in lymphoid cells, and thus be useful in the new methods. A 24p3R-like polypeptide containing at least 80% sequence identity, e.g., 85%, 90%, 95%, 98%, or 99%, with a 24p3R amino acid sequence is also useful in the new methods, as long as the resulting polypeptide measurably induces apoptosis in a lymphoid cell. Furthermore, a 24p3R polypeptide with up to 30, e.g., 1, 3, 5, 10, 15, 20, or 25, conservative amino acid substitutions; and up to 20, e.g., 1, 3, 5, 10 or 15, non-conservative amino acid substitutions or deletions (in any combination, e.g., 10 deletions and 10 substitutions) will be useful in methods of the invention as long as the resulting polypeptide measurably modulates apoptosis in a lymphoid cell. In the preceding sentence, “deletion” refers to one amino acid. Thus, “20 deletions” means the deletion of a total of 20 amino acid residues, which may or may not be consecutive.

The determination of percent identity between two amino acid sequences is accomplished using the BLAST 2.0 program, which is available to the public at ncbi.nlm.nih.gov/BLAST. Sequence comparison is performed using an ungapped alignment and using the default parameters (BLOSUM 62 matrix, gap existence cost of 11, per residue gap cost of 1, and a lambda ratio of 0.85). The mathematical algorithm used in BLAST programs is described in Altschul et al., 1997, Nucleic Acids Research, 25:3389-3402.

In certain embodiments, a 24p3R polypeptide used in the new methods is glycosylated. For example, the glycosyl moiety is N-linked to an amino acid residue in a mature 24p3R polypeptide (Rudd et al., 1999, Biochemistry, 38:13937-13950). A glycosylated 24p3R polypeptide can be obtained by purification of a naturally occurring 24p3R polypeptide from a suitable source, e.g., from humans, mice, or rats. Recombinant, glycosylated 24p3R polypeptides also can be produced by conventional methods, using transformed eukaryotic cells, e.g., yeast cells.

In some embodiments, a 24p3R polypeptide is modified by derivatization of amino acid side chains, chemical conjugation, or fusion to non-24p3R peptide moieties. For example a 24p3R amino acid sequence can be fused to an N-terminal peptide moiety or C-terminal peptide moiety, to increase in vivo serum half-life of the polypeptide. In some embodiments, the 24p3R polypeptide contains one or more modified amino acids, e.g., D-amino acids. Modified amino acids are useful for purposes such as increasing serum half-life of a polypeptide.

Production of 24p3R Polypeptides

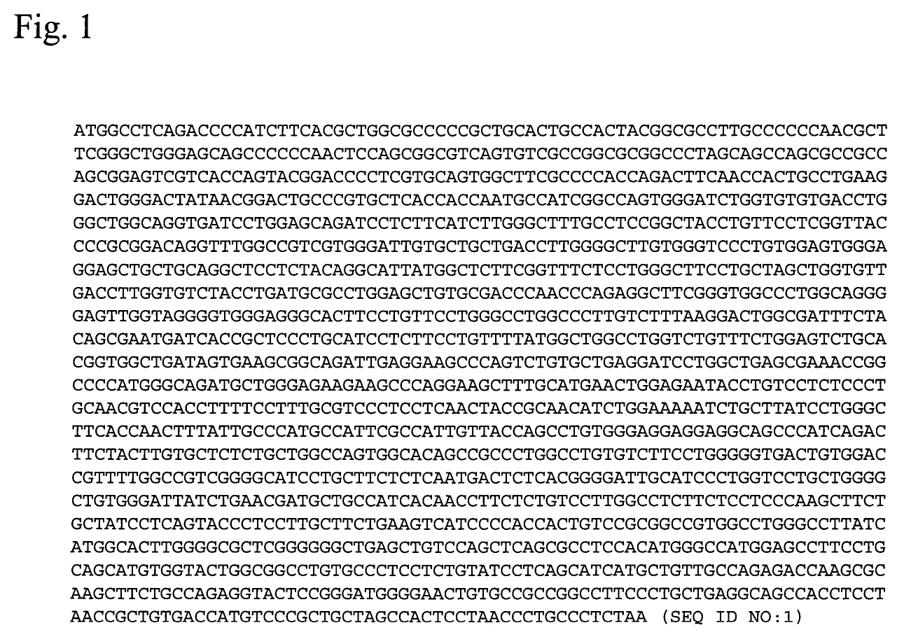

Polypeptides for use as described herein can be obtained by any suitable method. One method of producing a 24p3R polypeptide is recombinant production, which involves genetic transformation of a host cell with a recombinant nucleic acid vector encoding the polypeptide or pre-polypeptide, expression of the recombinant nucleic acid in the transformed host cell, and collection and purification of the 24p3R polypeptide. Guidance and information concerning recombinant DNA methods and materials for production of polypeptides can be found in numerous treatises and reference manuals, e.g., Sambrook et al., 1989, Molecular Cloning—A Laboratory Manual, 2^{nd} Ed., Cold Spring Harbor Press; Ausubel et al., (eds.), 1994, Current Protocols in Molecular Biology, John Wiley & Sons, Inc.; Innis et al., 1990, PCR Protocols, Academic Press. Nucleotide sequences are provided herein (FIG. 1 (SEQ ID NO:1), Genbank Accession No. NP_{—}803156, and Genbank Accession No. NP_{—}065105). For specific guidance concerning cloning of human 24p3R cDNA by PCR, and recombinant production of human 24p3R polypeptides, see Bundgaard et al., 1994, Biochem. Biophys. Res. Commun., 202:1468-1475; Bartsch et al., 1995, FEBS Lett., 357:255-259.

A 24p3R polypeptide also can be obtained directly by chemical synthesis, e.g., using a commercial peptide synthesizer according to the vendor's instructions. Methods and materials for chemical synthesis of polypeptides are well known in the art.

Alternatively, 24p3R polypeptides useful in the invention can be isolated from natural sources. For example human 24p3R can be isolated from human neutrophils using methods and materials known by those skilled in this field. Techniques for purification of 24p3R polypeptides from biological material are also known in this field. 24p3R is an integral membrane protein and can be purified using known techniques. In addition, as discussed further below, techniques for production of anti-24p3R antibodies are known, and the use of the antibodies in purification and assay of 24p3R polypeptides are also known. See, e.g., Kjeldsen et al., 1993, J. Biol. Chem., 268:10425-10432; Liu et al., 1997, Molecular Reproduction and Development, 46:507-514.

24p3R Antibodies

In another aspect, anti-24p3R antibodies are provided. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The new antibodies can be polyclonal, monoclonal, recombinant, e.g., chimeric or humanized, fully human, non-human, e.g., murine, or single chain antibodies. Methods of making such antibodies are known. In some cases, the antibodies have effector function and can fix complement. The antibodies can also be coupled to toxins, reporter groups, or imaging agents.

Full-length 24p3R polypeptides or proteins, or antigenic peptide fragments of 24p3R, can be used as an immunogen or can be used to identify anti-24p3R antibodies made with other immunogens, e.g., cells, membrane preparations, and the like. Antigenic peptides of 24p3R should include at least 8 amino acid residues of the full-length amino acid sequence, e.g., as shown in SEQ ID NO:2, and encompass an epitope of 24p3R. The antigenic peptides can include at least 10, 15, 20, or 30 amino acid residues.

Fragments of 24p3R that include residues at the C terminus (e.g., amino acid residues 428-520 of SEQ ID NO:2 or the C terminal 14 amino acids (CDHVPLLATPNPAL, SEQ ID NO:3)), N terminus (e.g., amino acid residues 1-153 of SEQ ID NO:2), or domains predicted to be intracellular can be used to make, e.g., used as immunogens or used to characterize the specificity of an antibody, antibodies against hydrophilic of the 24p3R protein. Similarly, fragments of 24p3R that include residues predicted to be transmembrane domains can be used to make an antibody against a hydrophobic region of the 24p3R protein. Antibodies reactive with, or that bind specifically to, any of these regions, or other regions or domains described herein are provided. These so-called fragments can also be used as the 24p3R polypeptides in the various methods described herein.

Epitopes encompassed by the antigenic peptides are generally regions of 24p3R located on the surface of the protein, e.g., hydrophilic regions, as well as regions with high antigenicity. For example, an Emini surface probability analysis of the human 24p3R protein sequence can be used to indicate the regions that have a particularly high probability of being localized to the surface of the 24p3R protein and are thus likely to constitute surface residues useful for targeting antibody production.

In some embodiments, the antibody can bind to the extracellular portion of the 24p3R protein, e.g., it can bind to a whole cell that expresses the 24p3R. In another embodiment, the antibody can bind to an intracellular portion of the 24p3R.

In another embodiment, the antibody binds an epitope on any domain or region on 24p3R proteins described herein.

Chimeric, humanized, e.g., completely human, antibodies are desirable for applications that include repeated administration, e.g., therapeutic treatment (and some diagnostic applications) of a human subject. Such antibodies may have activity that activates or inhibits either the iron transport function of 24p3R or the apoptotic signaling pathway that is mediated by 24p3R. Methods of determining the activity elicited by a particular antibody can be determined using methods known in the art and methods described herein.

The anti-24p3R antibody can be a single chain antibody. A single-chain antibody (scFV) may be engineered (for example, Colcher et al., 1999, Ann. N.Y. Acad. Sci., 880:263-280; and Reiter, 1996, Clin. Cancer Res., 2:245-252). The single chain antibody can be dimerized or multimerized to generate multivalent antibodies having specificities for different epitopes of the same target 24p3R protein.

In some cases, the antibody has reduced or no ability to bind to an Fc receptor. For example, it is an isotype or subtype, fragment or other mutant, which does not support binding to an Fc receptor, e.g., it has a mutagenized or deleted Fc receptor binding region.

An anti-24p3R antibody (e.g., monoclonal antibody) can be used to isolate a 24p3R by standard techniques, such as affinity chromatography or immunoprecipitation. Moreover, an anti-24p3R antibody can be used to detect a 24p3R (e.g., in a cellular lysate or cell supernatant) to evaluate the abundance and pattern of expression of the protein. Anti-24p3R antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Animal Models

Animal models can be used for testing 24p3R polypeptides, e.g., for their efficacy in treating a disorder, estimating toxicity, and dosages. Methods for performing such tests are known in the art. Suitable animal models include animal models for leukemias and autoimmune disorders, e.g., a Fas knockout mouse which exhibits an autoimmune lymphoproliferative syndrome (APLS). Mouse models for dysregulated iron metabolism are also useful and include beta2-microglobulin knockout (beta 2m −/−) mice, which develop iron overload; and mice deleted in the hfe gene (hfe −/−), which abnormally accumulate iron in tissues. Defects in the human hfe gene result in hemochromatosis.

Effective Dose

Toxicity and therapeutic efficacy of the new 24p3R polypeptides can be determined by standard pharmaceutical procedures, using either cells in culture or experimental animals to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Polypeptides that exhibit large therapeutic indices are preferred. While polypeptides that exhibit toxic side effects may be used, care should be taken to design a delivery system that targets such compounds to the site of affected tissue to minimize potential damage to non-target cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the new methods, the therapeutically effective dose can be estimated initially from cell culture assays. A dose can also be calculated in animal models to achieve a circulating plasma concentration range that includes the IC50 (that is, the concentration of the test compound which achieves a half-maximal inhibition of symptoms) as determined in cell culture. Such information can be used to more accurately determine useful doses in humans.

Formulation, Dosage and Administration

A 24p3R polypeptide, or a compound that binds to and affects the activity of a 24p3R polypeptide, can be administered according to the invention by any suitable method. Preferably, the polypeptide is administered parenterally, to avoid digestion in the stomach. Parenteral administration can be systemic, e.g., by an intravenous route. In some embodiments of the invention, the polypeptide is administered locally, e.g., into a tumor or lymph node.

The present invention provides pharmaceutical compositions for treating individuals in need of treatment for a lymphoid cell disease (e.g., a leukemia or autoimmune disorder). The treatment method entails administering a therapeutically effective amount of a 24p3R polypeptide or 24p3R-like polypeptide that causes apoptosis of a lymphoid cell and, optionally, a pharmaceutically acceptable carrier, diluent, excipient, or adjuvant. In certain embodiments, the polypeptide is administered by delivering a nucleic acid sequence to cells of the individual, where the nucleic acid encodes the corresponding polypeptide.

The pharmaceutical compositions can be used for humans or animals (e.g., mammals) and can include any one or more of a pharmaceutically acceptable diluent, carrier, excipient, or adjuvant. The choice of pharmaceutical carrier, excipient, and diluent can be selected with regard to the intended route of administration and standard pharmaceutical practice. The pharmaceutical compositions can include as (or in addition to) the carrier, excipient, or diluent, any suitable binder(s), lubricant(s), suspending agent(s), coating agent(s), or solubilizing agent(s).

The invention includes pharmaceutical formulations that include a pharmaceutically acceptable excipient and a 24p3R polypeptide or 24p3R-like polypeptide. Such pharmaceutical formulations can be used in a method of treating a lymphoid cell disorder such that at least one symptom of the disease is ameliorated. Such a method entails administering to the individual a therapeutically effective amount of the pharmaceutical formulation, i.e., an amount sufficient to ameliorate signs and/or symptoms of the lymphoid cell disease. In particular, such pharmaceutical formulations can be used to treat lymphoid cell disease in mammals such as humans and domesticated mammals (e.g., cows, pigs, dogs, and cats). The efficacy of such treatment can be estimated in an animal model system well known to those of skill in the art as discussed herein. Such a composition typically contains from about 0.1 to 90% by weight (such as 1 to 20% or 1 to 10%) of a 24p3R polypeptide or 24p3R-like polypeptide of the invention in a pharmaceutically acceptable carrier.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water-soluble versions of the compounds can be administered by the drip method, whereby a pharmaceutical formulation containing the 24p3R polypeptide or 24p3R-like polypeptide and a physiologically acceptable excipient is infused. Physiologically acceptable excipients can include, for example, 5% dextrose, 0.9% saline, Ringer's solution, or other suitable excipients. For intramuscular preparations, a sterile formulation of a suitable soluble salt form of the compounds can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid, (e.g., ethyl oleate). Oral or topical methods of delivery may be used. Such methods are known in the art.

The optimal percentage of the 24p3R polypeptide or 24p3R-like polypeptide in each pharmaceutical formulation varies according to the formulation itself and the therapeutic effect desired in the specific pathologies and correlated therapeutic regimens. Appropriate dosages of the 24p3R-polypeptide or 24p3R-like polypeptide can be determined by those of ordinary skill in the art of medicine by monitoring the individual, e.g., a mammal, for signs of disease amelioration or inhibition, and increasing or decreasing the dosage and/or frequency of treatment as desired. The optimal amount of the 24p3R polypeptide or 24p3R-like polypeptide used for treatment of lymphoid cell diseases depends upon the manner of administration, the age and the body weight of the subject, and the condition of the subject to be treated. Generally, the 24p3R polypeptide or 24p3R-like polypeptide is administered at a dosage of 1 to 100 mg/kg body weight, and typically at a dosage of 1 to 10 mg/kg body weight. In treatment of a lymphoid cell disorder such as a leukemia or immune disorder, dosage is adjusted so as to achieve an 24p3R polypeptide or 24p3R-like polypeptide serum concentration in the range of 0.1 ng/ml to 100 ng/ml, e.g., in the range of 1.0 ng/ml to 20 ng/ml, at least once every two weeks, e.g., once per week, once every third day, once every second day, or once per day. Such optimization is within ordinary skill in the art. The compound can also be administered chronically. The skilled artisan will appreciate that certain factors may influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a compound can include a single treatment or, preferably, can include a series of treatments.

Natural 24p3R is a membrane protein, and its target in the induction of apoptosis in lymphoid cells is believed to be exposed on the outside of the lymphoid cells, which occur in the blood and lymphatic system. Therefore, in the practice of the new methods, the 24p3R polypeptide need not cross cytoplasmic membranes or otherwise enter into cells, nor does it need to penetrate solid tissues to be effective.

A 24p3R polypeptide can be formulated into a pharmaceutical composition by admixture with pharmaceutically acceptable nontoxic excipients or carriers. Such compositions can be prepared for use in parenteral administration, particularly in the form of liquid solutions or suspensions. The composition can be administered conveniently in unit dosage form. Such methods are described, e.g., in Remington's Pharmaceutical Sciences, Mack Pub. Co., Easton, Pa. In some embodiments, the polypeptide is administered gradually, in a buffered saline solution, by intravenous infusion.

Methods of Modulating Iron Content

24p3R can be utilized to modulate the iron content of cells. 24p3R acts to import 24p3 ligand into cells. 24p3 ligand can exist in an iron-bound form, holo-24p3, or in an iron-free form, apo-24p3. Since holo-24p3 contains iron, delivering holo-24p3 intracellularly will increase the iron content of the cell. On the other hand, apo-24p3 acts as an iron chelator, therefore delivering apo-24p3 intracellularly will decrease the free iron concentration within the cell. By acting to import 24p3 into the cell, 24p3R modulates the iron content of cells depending on the iron-bound state of the 24p3 ligand. Iron-bound or iron-free 24p3 can be administered according to the invention by any suitable method. Preferably, the polypeptide is administered parenterally, to avoid digestion in the stomach. Parenteral administration can be systemic, e.g., by an intravenous route. In some embodiments of the invention, the polypeptide is administered locally, e.g., into a tumor or lymph node.

Methods of Modulating Apoptosis

24p3R can be utilized to modulate apoptosis of cells by modulating intracellular iron concentration. A decrease in intracellular free iron, e.g., by an iron chelator, can cause apoptosis of cells. Binding and uptake of holo-24p3 by 24p3R can increase intracellular iron availability and make cells resistant to apoptosis. Binding and uptake of apo-24p3 by 24p3R can chelate intracellular iron, decreasing iron availability and causing apoptosis.

Increasing the affinity of 24p3R for holo-24p3 (e.g., by administering a compound that increases the affinity) can lead to a decrease in apoptosis, whereas increasing the affinity of 24p3R for apo-24p3 (e.g., by administering a compound that increases the affinity) can lead to an increase in apoptosis. Decreasing the affinity of 24p3R for either form of the 24p3 ligand can have an effect opposite to those described above.

Iron-free 24p3 can be produced and administered parenterally, e.g., locally, e.g., by injection, to the tissue surrounding cancer cells that express 24p3R to induce apoptosis.

Gene therapy techniques can be used to introduce nucleic acids directing the expression of 24p3R to cancer cells that do not express 24p3R, or to increase the expression of 24p3R in cells that already express low levels of 24p3R. The nucleic acids described herein, e.g., nucleic acids encoding a 24p3R polypeptide or biologically active fragment thereof can be incorporated into a gene construct to be used as a part of a gene therapy protocol. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, 1990, Blood, 76:271).

A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM, which are known to those skilled in the art. Examples of suitable packaging virus lines for preparing both ecotropic and amphotropic retroviral systems include ΨCrip, ΨCre, Ψ2 and ΨAm.

Retroviruses have been used to introduce a variety of genes into many different cell types, including epithelial cells, in vitro and/or in vivo (see for example Eglitis, et al., 1985, Science, 230:1395-1398; Danos and Mulligan, 1988, Proc. Natl. Acad. Sci. USA, 85:6460-6464; Wilson et al., 1988, Proc. Natl. Acad. Sci. USA, 85:3014-3018; Armentano et al., 1990, Proc. Natl. Acad. Sci. USA, 87:6141-6145; Huber et al., 1991, Proc. Natl. Acad. Sci. USA, 88:8039-8043; Ferry et al., 1991, Proc. Natl. Acad. Sci. USA, 88:8377-8381; Chowdhury et al., 1991, Science, 254:1802-1805; van Beusechem et al., 1992, Proc. Natl. Acad. Sci. USA, 89:7640-7644; Kay et al., 1992, Human Gene Therapy, 3:641-647; Dai et al., 1992, Proc. Natl. Acad. Sci. USA, 89:10892-10895; Hwu et al., 1993, J. Immunol., 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Barker et al., 1998, BioTechniques, 6:616; Rosenfeld et al., 1991, Science, 252:431-434; and Rosenfeld et al., 1992, Cell, 68:143-155. Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., 1992, supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, 1986, J. Virol., 57:267.

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol., 158:97-129. It is also one of the few viruses that may integrate its DNA into non-dividing cells, and exhibits a high frequency of stable integration (see for example Flotte et al., 1992, Am. J. Respir. Cell. Mol. Biol., 7:349-356; Samulski et al., 1989, J. Virol., 63:3822-3828; and McLaughlin et al., 1989, J. Virol. 62:1963-1973). Vectors containing as little as 300 base pairs of AAV can be packaged and can integrate. Space for exogenous DNA is limited to about 4.5 kb. An AAV vector such as that described in Tratschin et al., 1985, Mol. Cell. Biol., 5:3251-3260 can be used to introduce DNA into cells. A variety of nucleic acids have been introduced into different cell types using AAV vectors (see for example Hermonat et al., 1984, Proc. Natl. Acad. Sci. USA, 81:6466-6470; Tratschin et al., 1985, Mol. Cell. Biol., 4:2072-2081; Wondisford et al., 1988, Mol. Endocrinol. 2:32-39; Tratschin et al., 1984, J. Virol. 51:611-619; and Flotte et al., 1993, J. Biol. Chem. 268:3781-3790.

In addition to viral transfer methods, such as those illustrated above, non-viral methods can also be employed to cause expression of a nucleic acid compound described herein (e.g., a polypeptide encoding 24p3R nucleic acid) in the tissue of a subject. Typically, non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., 2001, J. Invest. Dermatol., 116:131-135; Cohen et al., 2000, Gene Ther., 7:1896-905; or Tam et al., 2000, Gene Ther., 7:1867-74.

In some embodiments, a gene encoding a compound described herein, e.g., a 24p3R, is entrapped in liposomes bearing positive charges on their surface (e.g., lipofectins), which can be tagged with antibodies against cell surface antigens of the target tissue (Mizuno et al., 1992, No Shinkei Geka, 20:547-551; PCT publication WO91/06309; Japanese patent application 1047381; and European patent publication EP-A-43075).

In clinical settings, the gene delivery systems for the therapeutic gene can be introduced into a subject by any of a number of methods, each of which is known. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., 1994, Proc. Natl. Acad. Sci. USA, 91: 3054-3057).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

Modulation of cellular apoptosis can be useful in the treatment of cancer. Apoptosis can be increased in cancer cells, thus shrinking tumors. Alternately, apoptosis can be inhibited in the surrounding tissue. This can inhibit tumor growth by aiding immune cells, or, in contact-inhibited cancers, by decreasing the free space available in which the tumor can grow.

### EXAMPLES

The invention is further illustrated by the following examples. The examples are provided for illustrative purposes only. They are not to be construed as limiting the scope or content of the invention in any way.

Materials and Methods

Cell Lines

IL-3-dependent FL5.12, BaF3, 32D, and murine primary bone marrow (BM) cells, WEHI 7.2; IL-2-dependent HT-2 cells; IL-7-dependent D1-F4 cells; IL-6-dependent 7TD1 cells were cultured as described in Devireddy et al. (2001, Science, 293:829-834). COS-7, HeLa/NIH 3T3 cells were obtained from the American Type Culture Collection (ATCC) and cultured according to the supplier's instructions. Transfections were performed either by using SuperFect® (Qiagen) or Geneportemm (Gene Therapy Systems). Transfected cells were selected in G418 or blasticidin (Invitrogen).

Expression Cloning of the 24p3 Receptor

Poly (A)⁺ mRNA was isolated from mouse IL-3 dependent FL5.12 cells using an OligoTex mRNA kit (Qiagen, Valencia, Calif.) according to the manufacturer's instructions. A cDNA library was synthesized using an oligo-dT primer, and size-selected cDNAs (>1 kb) were then directionally cloned into the vector pCMB-SPORT6 (GIBCO-BRL). The cDNA clones were amplified in Luria-Bertani (LB) medium and the plasmid DNA was isolated using a Maxi Prep kit (Qiagen, Valencia, Calif.). Pooled purified plasmid DNAs were transfected into COS-7 cells. The transfected cells were dislodged in (phosphate buffered saline) PBS containing 0.5 mM EDTA, and resuspended in binding buffer (2% bovine serum albumin (BSA) in PBS). The cells were then panned on GST-24p3 coated plates (10⁷ cells/plate) that were prepared as follows. Bacteriological petri plates (Falcon) were layered with 10 μg/ml anti-mouse IgG (KPL) in 50 mM Tris-HCl, pH 9.5, rinsed with 0.15 M NaCl and blocked with BSA blocking buffer (KPL). Anti-GST monoclonal antibody (1 μg/ml) (Santa Cruz Biotechnology) in PBS/2% BSA was added to each dish and incubated overnight at 4° C. The plates were washed with PBS and incubated with 20 μg of purified GST-24p3. Non-adherent cells were removed by washing with PBS/2% BSA and the adherent cells were lysed with Hirt buffer (0.6% SDS, 10 mM Tris, 10 mM EDTA pH 7.5-8) and the plasmid DNA was recovered. The resulting plasmid DNA was transformed into XL 1-Blue competent cells (Stratagene) and amplified and the panning procedure was repeated two more times. After three rounds of panning, individual plasmids were purified and transfected into COS-7 cells and the transfected cells were screened for their ability to bind ³²P-24p3 (Devireddy et al., 2001, supra). Inserts from plasmids that conferred binding to ³²P-24p3 were sequenced. Full-length 24p3R cDNA was obtained from Image clone number 5365976 (American Type Culture Collection).

Purification of Recombinant 24p3

Full-length 24p3 cDNA was cloned into pGEX2TK vector (Pharmacia) and transformed into E. coli strain BL21 (Pharmacia). The bacteria were then grown in LB medium, or LB medium supplemented with 50 mM FeCl₂ (Sigma) or with 10 μM DFO (an iron chelator, deferoxamine mesylate; Calbiochem) and induced with 1 mM IPTG (isopropyl-beta-D-thiogalactopyranoside; Promega). The bacteria were lysed and the recombinant 24p3 was purified as described in Bundgaard et al., (1994, Biochem. Biophys. Res. Comm. 202:1468-1475).

Internalization Assay

HeLa cells constitutively expressing 24p3R were treated with ³²P-24p3 and at various time points cells were washed three times with PBS and incubated for 15 minutes in proteinase K, trypsin, and EDTA solution to remove the extracellular portion of the receptor. The dislodged cells were then collected and counted.

Iron Determination Procedure

Colorimetric determination of intracellular non-haem iron concentration was measured as described by Leardi et al., (1998, Br. J. Haematol., 102:746-752) with modifications. Briefly, cells were washed twice with ice-cold PBS and then lysed with lysis buffer (1% TRITON™-X100, 10% glycerol. 25 mM HEPES, pH 7.4, in PBS) and incubated with 0.3 N HCl. The proteins in the lysates were precipitated with 10% TCA (trichloroacetic acid) and centrifuged at 1200×g for 15 minutes. The lysates were then mixed with iron chromogen, FERROZINE® (Sigma) in iron buffer reagent (1.5% hydroxylamine HCl in acetate buffer, pH 4.5) and the absorbance was measured spectrophotometrically at 560 nm. The non-heme iron concentration was measured by interpolating the absorbance of each sample using preset iron standards (Sigma) and expressed as mg/dL.

Cyt ‘c’ extraction, cell-free extraction, and PARP extractions were performed using methods known in the art.

### Example 1

### Identification of a Murine 24p3 Receptor

To isolate a cell surface receptor for 24p3, expression closing was performed using a cDNA library prepared from mouse IL-3-dependent pro-B lymphocytic FL5.12 cells, which are highly sensitive to 24p3-mediated apoptosis (Devireddy et al., 2001, Science, 293: 829-834). The cDNA library was transfected into COS-7 cells and, after three rounds of panning, two clones were identified that conferred maximal 24p3 binding activity. These two clones contained cDNA inserts of about 1.1 and about 2.0 kb. DNA sequence analysis revealed that the two clones contained the same sequence, which showed significant similarity to a full-length mouse cDNA encoding a protein named brain type organic cation transporter (BOCT) (Genbank accession no. NM_{—}021551), now renamed 24p3R. This protein is also referred to as BOIT, and its approved gene symbol name is SLC22A17.

The full-length cDNA contained a single open reading frame of 520 amino acids (FIG. 2; SEQ ID NO:2). Hydropathy analysis predicted twelve putative transmembrane helices. A model of 24p3R, obtained by several computer programs (i.e., TOPRED program; von Heijne, 1992, J. Mol. Biol., 225:487-494), suggested a transmembrane topology in which both the N- and C-terminals are extracellular. Consistent with this prediction, the 24p3-interacting clones contained only the C-terminal portion and the last two transmembrane regions of 24p3R, indicating that the C terminal region interacts with 24p3. Accordingly, the N and C termini, and in particular the C terminus, of a 24p3 are useful as targets for compounds that bind to 24p3R and can affect function. An example of such a compound is an antibody the specifically binds to the N or C terminus of a 24p3.

### Example 2

### Expression of 24p3R

To determine the expression pattern of 24p3R, a polyclonal anti-24p3R antibody was raised using as an immunogen a peptide from 24p3R that contained the C terminal 14 amino acids of 24p3R (CDHVPLLATPNPAL, SEQ ID NO:3). The antibody detected a 60 kDa protein in hematopoietic cells, consistent with the predicted size of 24p3R. Detection of the 60 kDa polypeptide was blocked by incubation of the antiserum with the peptide immunogen, but was not blocked by an irrelevant peptide.

Immunoblot analysis on a panel of murine tissues revealed that 24p3R was expressed ubiquitously. Several tissues, such as spleen and testis, had a smaller, approximately 30 kDa form of 24p3R. RT-PCR analysis indicates that this shorter form represents an alternatively spliced variant that lacks the N-terminal 154 amino acids, but contains the C-terminal region that interacts with 24p3. These forms are referred to herein as 24p3R-long (24p3R-L) and 24p3R-short (24p3R-S). Immunoblot analysis of a panel of cell lines revealed that 24p3R was expressed to varying extents in some, but not all, cells with different 24p3R-L/24p3R-S ratios. The 24p3R-S isoform appears to have a higher affinity for 24p3. RT-PCT analysis during different stages of mouse development revealed that 24p3R was expressed as early as day seven and persisted at least until embryonic day 17.

### Example 3

### 24p3R Function

HeLa cells expressed only low levels of 24p3R. To perform functional experiments HeLa cell lines were derived that stably express recombinant N-terminal epitope-tagged derivatives of 24p3R-L and 24p3R-S (HeLa/24p3R-L and HeLa/24p3R-S) and, as a control, the empty expression vector (HeLa/X7). Immunoblot analysis confirmed expression of 24p3R in HeLa/24p3R-L and HeLa/24p3R-S cells.

To determine whether the ectopically expressed 24p3R was able to bind 24p3, a series of ligand-cell binding experiments were performed. It was found that ³²P-labeled 24p3 bound to HeLa/24p3R-L and HeLa/24p3R-S cells, but not to HeLa/X7 cells. Binding of ³²P-labeled 24p3 to HeLa/24p3R-L and HeLa/24p3R-S was saturable and blocked by the addition of an excess of unlabeled 24p3. Scatchard analysis indicated that 24p3 bound to the receptor in a 1:1 complex with an affinity of approximately 100 pmol. Thus, ectopically expressed 24p3R can bind to 24p3.

24p3 and other lipocalins are internalized following binding to their cell surface receptor. Consistent with this, ³²P-labeled 24p3 was internalized by HeLa/24p3R-L and HeLa/24p3R-S cells, but not by HeLa/X7 cells. This further demonstrates that ectopically expressed 24p3R functions correctly. Thus, cells ectopically expressing 24p3R can be used as reagents for examining the function of 24p3 and 24p3R as well as in assays to identify compounds that affect expression or activity of a 24p3R or a 24p3 (e.g., 24p3 or NGAL).

### Example 4

### 24p3R and Iron Delivery

Experiments were performed to determine whether 24p3R expression would confer on cells the ability to deliver iron in a 24p3-dependent fashion. In these experiments, as well as those described below, three preparations of 24p3 were used: bacterially derived iron-loaded 24p3 (holo-24p3), bacterially derived iron-lacking 24p3 (apo-24p3), and conditioned medium (CM) from cytokine-deprived FL5.12 cells, which contains 24p3 (FL5.12-24p3). The three preparations of 24p3 were added to HeLa/24p3R-L, HeLa/24p3R-S, or HeLa/X7 cells, and, as a control, iron-containing transferrin (holo-transferrin) was added in parallel samples. To monitor intracellular iron concentration, the expression of two iron-regulated genes, ferritin and transferrin receptor 1 were examined.

The results of these experiments demonstrated that, in the presence of holo-transferrin, all three HeLa cell lines had increased iron concentration. This was demonstrated in all three HeLa cell lines by increased levels of ferritin and decreased levels of transferrin receptor 1. Holo-24p3 increased intracellular iron concentration in HeLa/24p3R-L and HeLa/24p3R-S cells, but not in HeLa/X7 cells. By contrast, the addition of apo-24p3 and FL5.12-24p3 did not increase intracellular iron in any of the HeLa cell lines. These data demonstrate that 24p3R is involved in the increase in intracellular iron concentration that is observed in the presence of holo-24p3. Thus, compounds that disrupt the interaction between 24p3R and holo-24p3 are useful for preventing an undesirable increase in intracellular iron.

To confirm the results disclosed above, a calorimetric assay that directly measures free iron concentration was employed. The results confirm that the addition of holo-24p3 increased intracellular iron in HeLa/24p3R-L and HeLa/24p3R-S cells, but not in HeLa/X7 cells. Unexpectedly, addition of apo-24p3 and FL5.12-24p3 substantially reduced free iron concentration in HeLa/24p3R-L and HeLa/24p3R-S cells. From these data, it can be concluded that 24p3R can mediate intracellular iron delivery upon addition of holo-24p3. Furthermore, the similar results obtained with apo-24p3 and FL5.12-24p3 suggest that FL5.12-24p3 lacks iron.

### Example 5

### 24p3R and Apoptosis

To examine the ability of 24p3R to mediate 24p3-dependent apoptosis, different preparations of 24p3 were added to the three HeLa cell lines described above and apoptosis was analyzed by annexin V-FITC staining. It was found that apo-24p3 and FL5.12-24p3 induced apoptosis in HeLa/24p3R-L and HeLa/24p3R-S cells, but not in HeLa/X7 cells. Apoptosis was confirmed by analyzing proteolytic cleavage of PARP (poly(ADP-ribose)polymerase) and caspase 3. In contrast, the addition of holo-24p3 failed to induce apoptosis in HeLa/24p3R-L and HeLa/24p3R-S cells.

It is well established that iron chelators can induce apoptosis. The combined results of the experiments discussed above suggest that apo-24p3 and FL5.12-24p3 induced apoptosis by acting as iron chelators to lower intracellular iron concentration. To examine this possibility, the expression of two genes, HIFIα and Nip3, was analyzed. The two genes are unregulated in response to decreased intracellular iron concentration. As a control, cells were treated with the bacterial siderophore desferrioxamine (DFO), an iron chelator, and analyzed in parallel. The results of these experiments show that apo-24p3 and FL5.12-24p3 increased HIFα levels and Nip3 transcription in HeLa/24p3R-S cells, but not in HeLa/X7 cells. As expected, DFO induced expression of HIFIα and Nip3 in all three HeLa cell lines.

The above results indicate that in cells expressing 24p3R, apo-24p3 and FL5.12-24p3 decrease intracellular iron levels and induce apoptosis. If the basis of 24p3-mediated apoptosis is in fact decreased intracellular iron concentration, then apoptosis should be prevented by iron delivery. To test this prediction, the ability of holo-transferrin to block 24p3-mediated apoptosis was examined. The results of these experiments indicated that holo-transferrin blocked apoptosis by 24p3, but had no effect on apoptosis induced by other agents, such as etoposide or staurosporine.

These data demonstrate that contacting a cell that expresses a 24p3R with holo-transferrin can provide a method of preventing apoptosis, whereas contacting a cell that expresses a 24p3R with apo-transferrin can provide a method of inducing apoptosis.

### Example 6

### Decreased Intracellular Iron Levels in HeLa Cells Expressing 24p3 Receptor Upon Treatment with apo-24p3

In this particular example, intracellular iron levels were measured in living cells using the iron-sensing dye calcein. Calcein is a fluorescent, cell permeable dye. Because its fluorescence is quenched upon iron binding, calcein can be used to measure free (also called labile or chelatable) iron in living cells. In other words, an increase in intracellular iron levels causes a decrease in fluorescence intensity, whereas a decrease in intracellular iron levels causes an increase in fluorescence intensity.

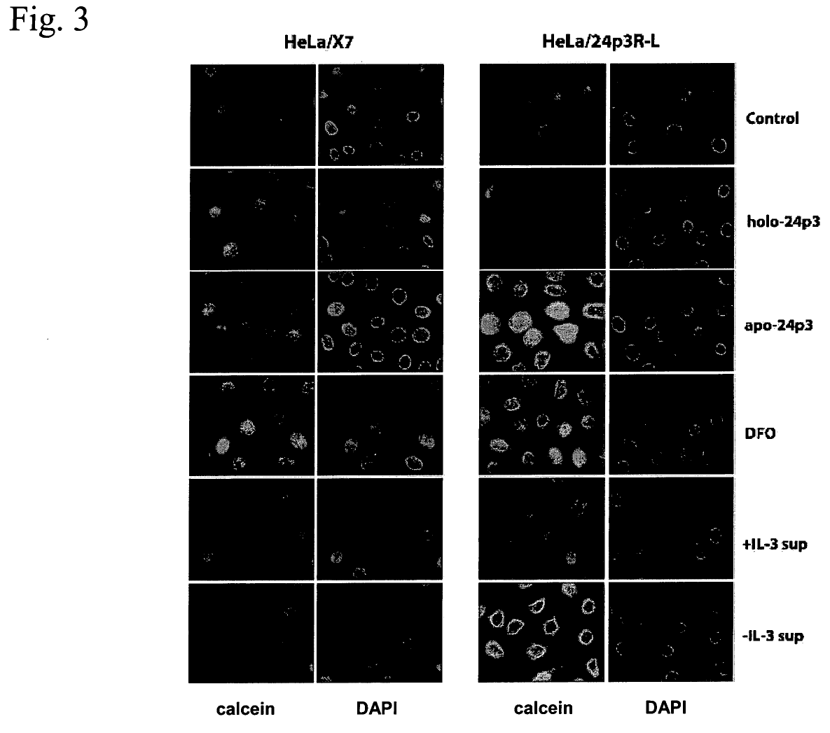

FIG. 3 shows that in calcein-loaded HeLa cells expressing 24p3 receptor there was a large increase in fluorescence emission upon treatment with any of apo-24p3, the known chemical iron chelator DFO, and conditioned medium from IL-3 withdrawn cells (−IL-3 Sup; contains 24p3), indicative of decreased intracellular iron levels. On the other hand, no change in intracellular iron levels were noticed upon treatment with control conditioned medium (+IL-3 sup; does not contain 24p3), although there was a decrease in fluorescence intensity upon treatment with holo-24p3, suggesting that holo-24p3 was able to deliver iron to the cells.

Control HeLa cells (HeLa/X7) did not exhibit any altered iron levels except for treatment with chemical iron chelator DFO. DFO mediated iron chelation is not dependent on the expression of 24p3 receptor.

This example further demonstrates that apo-24p3 can reduce intracellular iron concentration in cells that express 24p3R.

### Example 7

### BCR-ABL Counteracts the 24p3/24p3R Proapoptotic Pathway

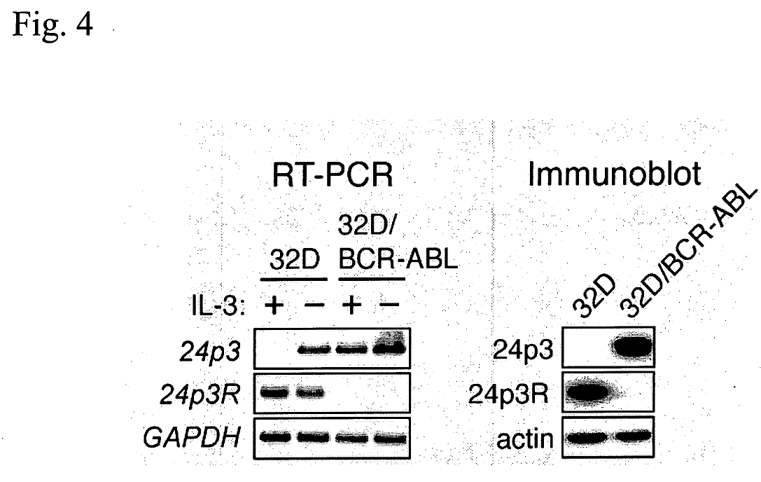

The mouse cell line 32D requires the cytokine IL-3 as a growth factor to prevent apoptosis. The cell line 32D/BCR-ABL^{p190}, which is the 32D cell line transfected with BCR-ABL^{p190}, is not dependent on IL-3. IL-3 deprivation induced apoptosis in 32D cells, but not in 32D/BCR-ABL^{p190} cells. To examine the involvement of 24p3 and 24p3R in this system, the expression of 24p3 and 24p3R was determined by RT-PCR and immunoblot analysis. FIG. 4 shows that in 32D cells 24p3 was expressed only following IL-3 withdrawal. Unexpectedly, in 32D/BCR-ABL^{p190} cells, 24p3 was constitutively expressed in the presence or absence of IL-3. 24p3R was expressed in 32D cells in the presence or absence of IL-3. However, in 32D/BCR-ABL^{p190} cells, 24p3R expression was dramatically down-regulated and unaffected by IL-3.

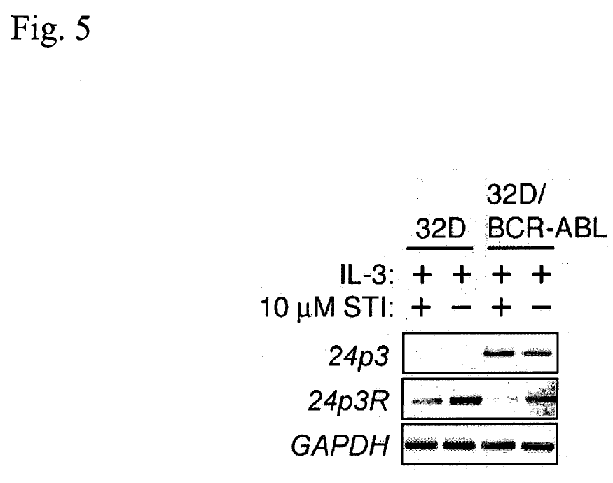

32D/BCR-ABL^{p190} cells that do not express 24p3R are unable to internalize 24p3. A cellular ligand-uptake experiment was performed in which 24p3 was fluorescently derivatized, added to 32D cells or 32D/BCR-ABL^{p190} cells, and 24p3 internalization analyzed by fluorescence microscopy. 32D cells, but not 32D/BCR-ABL^{p190} cells, internalized fluorescently labeled 24p3. Furthermore, conditioned medium from 32D/BCR-ABL^{p190} cells, but not from 32D cells, induced apoptosis when added to naïve, parental 32D cells, indicating that conditioned medium from 32D/BCR-ABL^{p190} cells contains 24p3. Finally, inhibition of BCR-ABL by STI (Gleevec) activated 24p3R expression in 32D/BCR-ABL^{p190} cells (FIG. 5).

These results indicate that expression of 24p3R may be useful for inducing apoptosis in a cancer cell.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying a candidate compound that modulates an interaction between a 24p3 polypeptide and a 24p3 receptor (24p3R) polypeptide, the method comprising
providing a sample comprising a 24p3R polypeptide;
contacting the sample with a 24p3 polypeptide and a test compound, thereby providing a test sample; and
determining whether binding between the 24p3 polypeptide and the 24p3R polypeptide is altered in the test sample compared to a control sample lacking the test compound, wherein a difference in binding indicates that the test compound is a candidate compound that modulates an interaction between the 24p3 polypeptide and the 24p3R polypeptide.

2. The method of claim 1, wherein the test compound decreases binding of the 24p3 polypeptide to the 24p3R polypeptide.

3. The method of claim 1, wherein the test compound increases binding of the 24p3 polypeptide to the 24p3R polypeptide.

4. The method of claim 1, wherein the test compound decreases an inflammatory response in an animal model

5. The method of claim 1, wherein the test compound decreases apoptosis in a cell.

6. The method of claim 1, wherein the test compound increases apoptosis in a cell.

7. The method of claim 1, wherein the test compound decreases iron transport in a cell.

8. The method of claim 1, wherein the test sample is a cell.

9. The method of claim 1, wherein the test sample is cell-free.

10. The method of claim 1, wherein the test sample comprises a mammalian cell.

11. The method of claim 1, wherein the test sample is derived from a mouse or a human.

12. The method of claim 1, wherein the test compound is an antibody.

13. The method of claim 1, wherein the test compound is a molecule that can bind to iron.

14. The method of claim 1, wherein the test compound is a 24p3 polypeptide.

15. A method of inducing apoptosis in a cell that expresses a 24p3 receptor (24p3R) polypeptide, the method comprising administering to the cell an amount of apo-24p3 sufficient to induce apoptosis.

16. The method of claim 15, wherein the cell is a mammalian cell.

17. The method of claim 15, wherein the cell is a human cell.

18. The method of claim 15, wherein the cell is in vivo.

19. The method of claim 15, wherein the cell is a cancer cell.

20. The method of claim 19, wherein the cancer cell is within a mammal.

21. The method of claim 20, wherein the mammal is a human.

22. A method of increasing 24p3 receptor (24p3R) polypeptide expression in a cell, the method comprising administering to the cell an expression vector comprising an isolated nucleic acid that codes for a 24p3R operably linked to an expression control sequence.

23. The method of claim 22, wherein the cell is in vitro.

24. The method of claim 22, wherein the cell is in vivo.

25. The method of claim 22, wherein the cell is a mammalian cell.

26. The method of claim 22, wherein the cell is a human cell.

27. The method of claim 22, wherein the cell is a cancer cell.

28. The method of claim 27, wherein the cancer cell is within a mammal.

29. An isolated nucleic acid molecule consisting of a nucleotide sequence that encodes a long form of a murine 24p3 receptor (24p3R-L) polypeptide having an amino acid sequence of SEQ ID NO:2, or the amino acid sequence of 24p3R-L with conservative amino acid substitutions.

30. The isolated nucleic acid of claim 29, wherein the nucleic acid consists of the nucleotide sequence of 24p3R-L (SEQ ID NO:1).

31. A purified polypeptide having the amino acid sequence of SEQ ID NO:2 with from 0 to 10 conservative amino acid substitutions.

32. The purified polypeptide of claim 31, wherein the polypeptide consists of the amino acid sequence of SEQ ID NO:2.

33. An expression vector comprising a nucleic acid of claim 29 operably linked to an expression control sequence.

34. A cultured cell comprising the expression vector of claim 33.

35. An antibody that specifically binds to a purified polypeptide of claim 31.

36. The antibody of claim 35, that specifically binds to an N-terminus or C-terminus of the polypeptide.

37. The antibody of claim 35, wherein the antibody binds to an N-terminus consisting of amino acid residues 1-153 of SEQ ID NO:2, or a portion thereof.

38. The antibody of claim 35, wherein the antibody binds to a C-terminus consisting of amino acid residues 428-520 of SEQ ID NO:2, or a portion thereof.

39. The antibody of claim 35, wherein the antibody specifically binds to the sequence CDHVPLLATPNPAL (SEQ ID NO:3).

40. The antibody of claim 35, wherein the antibody is a polyclonal antibody.

41. The antibody of claim 35, wherein the antibody is a monoclonal antibody.


SEQUENCE LISTING (3 sequences)

SEQ ID NO: 1 - DNA, 1563 bp, Mus musculus
atggcctcagaccccatcttcacgctggcgcccccgctgcactgccactacggcgccttgccccccaacgcttcgggctgggagcagccccccaactccagcggcgtcagtgtcgccggcgcggccctagcagccagcgccgccagcggagtcgtcaccagtacggacccctcgtgcagtggcttcgccccaccagacttcaaccactgcctgaaggactgggactataacggactgcccgtgctcaccaccaatgccatcggccagtgggatctggtgtgtgacctgggctggcaggtgatcctggagcagatcctcttcatcttgggctttgcctccggctacctgttcctcggttaccccgcggacaggtttggccgtcgtgggattgtgctgctgaccttggggcttgtgggtccctgtggagtgggaggagctgctgcaggctcctctacaggcattatggctcttcggtttctcctgggcttcctgctagctggtgttgaccttggtgtctacctgatgcgcctggagctgtgcgacccaacccagaggcttcgggtggccctggcaggggagttggtaggggtgggagggcacttcctgttcctgggcctggcccttgtctttaaggactggcgatttctacagcgaatgatcaccgctccctgcatcctcttcctgttttatggctggcctggtctgtttctggagtctgcacggtggctgatagtgaagcggcagattgaggaagcccagtctgtgctgaggatcctggctgagcgaaaccggccccatgggcagatgctgggagaagaagcccaggaagctttgcatgaactggagaatacctgtcctctccctgcaacgtccaccttttcctttgcgtccctcctcaactaccgcaacatctggaaaaatctgcttatcctgggcttcaccaactttattgcccatgccattcgccattgttaccagcctgtgggaggaggaggcagcccatcagacttctacttgtgctctctgctggccagtggcacagccgccctggcctgtgtcttcctgggggtgactgtggaccgttttggccgtcggggcatcctgcttctctcaatgactctcacggggattgcatccctggtcctgctggggctgtgggattatctgaacgatgctgccatcacaaccttctctgtccttggcctcttctcctcccaagcttctgctatcctcagtaccctccttgcttctgaagtcatccccaccactgtccgcggccgtggcctgggccttatcatggcacttggggcgctcggggggctgagctgtccagctcagcgcctccacatgggccatggagccttcctgcagcatgtggtactggcggcctgtgccctcctctgtatcctcagcatcatgctgttgccagagaccaagcgcaagcttctgccagaggtactccgggatggggaactgtgccgccggccttccctgctgaggcagccacctcctaaccgctgtgaccatgtcccgctgctagccactcctaaccctgccctctaa

SEQ ID NO: 2 - protein, 520 aa, Mus musculus
MASDPIFTLAPPLHCHYGALPPNASGWEQPPNSSGVSVAGAALAASAASGVVTSTDPSCSGFAPPDFNHCLKDWDYNGLPVLTTNAIGQWDLVCDLGWQVILEQILFILGFASGYLFLGYPADRFGRRGIVLLTLGLVGPCGVGGAAAGSSTGIMALRFLLGFLLAGVDLGVYLMRLELCDPTQRLRVALAGELVGVGGHFLFLGLALVFKDWRFLQRMITAPCILFLFYGWPGLFLESARWLIVKRQIEEAQSVLRILAERNRPHGQMLGEEAQEALHELENTCPLPATSTFSFASLLNYRNIWKNLLILGFTNFIAHAIRHCYQPVGGGGSPSDFYLCSLLASGTAALACVFLGVTVDRFGRRGILLLSMTLTGIASLVLLGLWDYLNDAAITTFSVLGLFSSQASAILSTLLASEVIPTTVRGRGLGLIMALGALGGLSCPAQRLHMGHGAFLQHVVLAACALLCILSIMLLPETKRKLLPEVLRDGELCRRPSLLRQPPPNRCDHVPLLATPNPAL

SEQ ID NO: 3 - protein, 14 aa, Mus musculus
CDHVPLLATPNPAL